(19) United States
(12) Patent Application Publication
(10) Pub. No.: US 2026/0294844 A1
(43) Pub. Date: Oct. 1, 2026

(54) THE INTEGRATED PLACEBO-SUBSTITUTE METHOD

(71) Applicant: Casie Corinne Alexander, Aurora, IL (US)

(72) Inventor: Casie Corinne Alexander, Aurora, IL (US)

(21) Appl. No.: 19/090,479

(22) Filed: Mar. 26, 2025

(51) Int. Cl. A61K 31/137; A61K 9/68; A61K 31/465

(52) U.S. Cl. CPC A61K 31/137; A61K 9/0058; A61K 31/465

(57) ABSTRACT

The Integrated Placebo-Substitute Method separately treats physical addiction and psycho-behavioral dependency. It involves two tools: (1) a controlled chemical substitute to satisfy physical cravings and (2) a nontoxic placebo to mimic addictive behavior. Individuals first use the substitute for physical withdrawal, then transition to the placebo when habits, rituals, or emotions trigger psychological cravings. By distinguishing between physical and psycho-behavioral needs, individuals gradually reduce substance use without experiencing withdrawal or deprivation, preventing relapse triggers like longing or romanticizing past use. This method allows for the restructuring of psychological responses after physical addiction is resolved. Designed for those who relapse post-withdrawal, this approach prioritizes both mind and body, offering a mindful, holistic alternative to traditional recovery methods.

### FIELD

The present application relates to a medical process; a sequential method that utilizes specific processes and tools to treat and heal addiction, as understood as a physical and psycho-behavioral phenomenon.

### BACKGROUND

Currently the most widely accepted and popular method for quitting addictive substances is the “cold turkey,” method, which means abruptly and completely stopping the addictive substance, suffering through a period of withdrawal, and coming out the other side, assumingly a healed individual. Alternatives to ‘cold turkey’ cessation are most often prescription drugs which aid the patient to physically replace and wean off the chemical dependency. Although there is considerable research on addiction, there are not developed treatment methods that are clearly defined and that differ considerably from these popular choices.

An obvious limitation of the cold turkey method is that it places the individual in a state of physical shock, most often called withdrawal. What many do not understand is this places the body and mind at odds with each other. During withdrawal, the body and the mind are in a constant state of betrayal in relation to the other; what the body craves the mind is tasked with depriving it of, and what the mind desires the body constantly spoils. Efforts toward sobriety are a hard battle, fought every minute of every day, and when relapse occurs, it becomes an internal degradation of oneself, by oneself.

However, there is even more to this picture. Individuals moving through the cold turkey method are suffering while in a physical state of shock and while their mind and body are at war with one another, but additionally, they are placed in a state of psycho-behavioral shock because they are simultaneously stripped of the habits, rituals, social and emotional uses, lifestyle, and even identity, that the addiction once provided them. Individuals are placed in a state of shock while effectively stripped of their coping mechanisms. While that may be conducive to change for some, for many it simply sets them up for an uphill battle at best, and relapse and failure at worst.

Without first acknowledging the fact that substances are used habitually, ritually (socially), and as coping mechanisms (emotionally), it is impossible to directly address the psychological relationship between the addicted person and their substance use. Cold turkey methods and replacement therapy methods alike position addicted persons to psychologically miss and even grieve their substances of choice, as they are also losing a part of their day-to-day life and behaviors. Psychologically, this creates a void to fill, and is responsible for relapse, even years after completing withdrawal. Psychologically, this creates a “dry drunk” scenario where individuals never fully resolve their attachment. Addicted persons may glorify or romanticize their addiction in the absence of using their substance because, in addition to being physically addicted to the addictive chemical, they are psychologically attached to the substance via their identity, beliefs, emotions and behaviors relating to it.

### SUMMARY

By developing a a medical process that is a sequential method which utilizes specific processes and tools to treat and heal addiction, as understood as a physical and psycho-behavioral phenomenon, the following issues are resolved:

Individuals are not placed into a state of physical or psychological withdrawal while their physical addiction (re: chemical dependency) is becoming resolved.

Individuals are able to restructure and resolve their psycho-behavioral dependency (re: habits, rituals, coping mechanisms, etc.) while their physical equilibrium is maintained by not experiencing any chemical addiction.

The Method consists of a two-Phase, sequential process where two key tools are utilized for different purposes: a chemical substitute and a placebo. In Phase I, the individual utilizes both tools.

A chemical substitute is used to respond to physical cravings and a placebo is used to respond to psycho-behavioral desires to use. In this way the individuals' physical and psycho-behavioral needs are both met. As psycho-behavioral desires are treated by the placebo, the addictive chemical present in the bloodstream of the individual is gradually reduced to nothing at the natural pace set by the interplay of the individuals' psycho-behavioral and physiological system.

Once the physical addiction or chemical dependency is completely resolved, the individual begins Phase II. During Phase II only the placebo is used as the individual only has the need to respond to psycho-behavioral needs. Free from the grip of chemical addiction, during this Phase, the individual gradually forms new, replacement habits, rituals, and coping mechanisms as they restructure their psycho-behavioral way of life. The use of the placebo is gradually reduced to nothing at the natural pace set by the individuals' psycho-behavioral system.

This Method was developed with the intention of helping addicts who continue to relapse, even after successfully completing the withdrawal process. Unlike other methods, it is a mindful, holistic, and comprehensive approach to addiction recovery where the needs of the mind and body are centered and prioritized.

### BRIEF DESCRIPTION OF THE DRAWINGS

In the following detailed portion of the present description, the teachings of the present application will be explained in more detail with reference to the example embodiments shown in the drawings, in which:

FIG. 1 is a schematic diagram embodiment illustrating the elements and sequence of the Method, its two Phases, and their elements, using abstract, graphic symbols.

FIG. 2 is a simple bar chart embodiment depicting change in utilization as a response to physical and psycho-behavioral needs over time.

### DETAILED DESCRIPTION

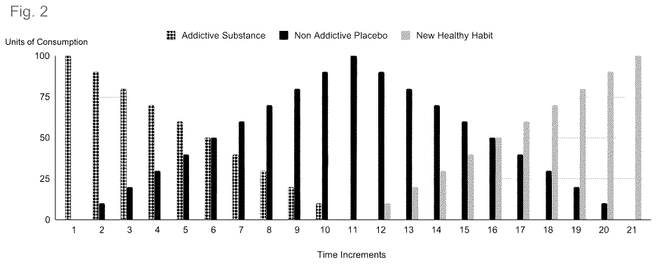

The Integrated Placebo-Substitute Method is a medical process of treating addiction. The Method is best understood as a two-phase process wherein a short series of steps and specific tool utilization is used to treat and heal addiction. The Method presupposes that addiction is understood as both a physical chemical addiction and a psycho-behavioral dependency, further consisting of both needs and desires. The Method therefore treats both the physical body and the psycho-behavioral system, but does so separately, in different ways, and at different times through the healing process. During Phase I, individuals resolve their chemical addiction using the help of a substitute, and during Phase II individuals resolve their psycho-behavioral dependency using the help of a placebo. That process is illustrated in FIG. 2.

Individuals must become keenly aware of the distinction between their physical and psycho-behavioral needs in real time in order to respond appropriately and to successfully utilize this Method without adverse effects. This Method is recommended to be administered by professionals in a controlled setting for best results and to ensure the treatment plan is understood and followed. Individuals may follow The Method in a semi-controlled setting or on their own but at their own risk, and only after receiving a disclaimer of the possible risks and adverse effects.

Before commencing The Method individuals must have a firm grasp on the practice of Mindfulness in order to develop the skill necessary to identify and distinguish between a physical craving and a psycho-behavioral need or desire.

At the beginning of The Method, (in Phase I), physical cravings and psycho-behavioral needs and desires will overlap. It is imperative to notice the precise moment that the physical craving is satisfied, so that the individual switches to using the placebo. It is only by this precise timing does The Method begin to succeed in gently weaning the individual from the addictive chemical. Failure to follow this procedure may not lead to addiction cessation and may lead to adverse effects.

To a lesser degree, it is also necessary to observe the individual's baseline consumption of the addictive substance over the course of at least one full week, before commencing The Method. The objective is to begin the Method while consuming the same amount of addictive substance substitute, so that the physical body does not experience deprivation, withdrawal or shock, and changes are both gradual and gentle. Any deprivation, whether of physical or psycho-behavioral nature, may produce a void to fill, resulting in warped efficacy and compromised results. Individuals should move through the changes of The Method while deeply connecting with and understanding their bodily needs and psycho-behavioral needs, without setting strict limits or timeline goals. Deprivation-real or perceived-may induce longing, romanticizing, or missing the substance, the associated lifestyle, or the identity of the individual, and compromise results. Strong addictions in individuals likely took years to solidify, so timelines should not be imposed on the healing process (in non-life-threatening circumstances).

Adequate precautions should be made should this Method be applied to addictions of dangerous degree, such as to treat Heroin addiction using the substitute Methadone with placebos. Such applications of The Method should always be made in a clinical setting and closely monitored by healthcare professionals, and only after sufficient clinical trials for such applications are successfully completed.

Other applications may require a lesser degree of precaution and oversight, such as applying The Method to tobacco or nicotine use, or even sugar addiction. However, the firm grasp and practice of Mindfulness and discernment between physical cravings and psycho-behavioral needs is still required in these applications, although the addictions may be less risky and/or morbid.

When both the preconditions are met of acquiring Mindfulness as a skill, and recording the individual's baseline consumption, Phase I of the Method may be commenced.

Phase I begins when the individual starts to identify and respond to physical cravings and psycho-behavioral needs and desires differently. A chemical substitute is used to respond to physical cravings and a placebo is used to respond to psycho-behavioral desires to use. In this way the individuals' physical and psycho-behavioral needs are still both being met, albeit separately and differently, instead of responding to both with the same addictive substance. Initially the substitute and placebo will be used during once sitting: individuals will use the substitute until the physical craving subsides, then if desire to use persists, they will switch to using the placebo until their psycho-behavioral need or desire completely subsides.

In the case of a Heroin addiction, Methadone would be a substitute. In the case of Tobacco addiction, Nicotine gum or another nicotine product may be used. In the example of Tobacco addiction, a non-addictive herbal cigarette may be used as a placebo. The purpose of the placebo is to mimic the behavior of the addiction without adding addictive chemicals into the bloodstream. During this phase of the Method, individuals are encouraged to consume as much of the placebo as they like so as to nurse the mind while the body is coming off the addiction.

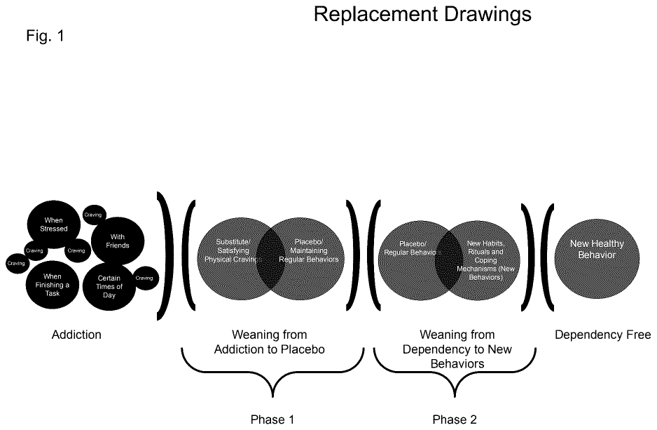

Because individuals are no longer responding to both physical cravings and psycho-behavioral needs and desires with the same addictive substance, and because isolating the addictive substance (or substitution) from the psycho-behavioral dependency would over time result in less and less addictive substance in the bloodstream, gradually, the patient would increasingly identify the desire to use, rather than the craving. The individual may then have more separation between the cravings and psycho-behavioral wants and needs, and so at some times during the day they may just use the substitute, and other times just use the placebo. Over time, the individual would fully wean themselves off of the chemical substitute at the natural pace set by the interplay of the individuals' psycho-behavioral and physiological system. This process is illustrated in FIG. 1, where Phase I is labeled. This marks the end of Phase I of the Method, wherein the physical addiction is resolved, and only the psycho-behavioral dependency remains.

Phase II begins once the physical addiction or chemical dependency is completely resolved. During Phase II only the placebo is used, as the individual only has the need to respond to psycho-behavioral needs. Free from the grip of chemical addiction, during this Phase, the individual gradually forms new, replacement habits, rituals, and coping mechanisms as they restructure their psycho-behavioral way of life. The use of the placebo is gradually reduced to nothing at the natural pace set by the individuals'psycho-behavioral system, over time. This process is illustrated in FIG. 1, Phase II.

Acute addictions in individuals likely took years to solidify, so timelines should not be imposed on the healing process in order to allow them to emotionally let go of habits, rituals, coping mechanisms, social practices and other psycho-behavioral roles the addiction once fulfilled for the individual. Rushing this process or setting restrictive goals or timelines may compromise results by inducing feelings of deprivation and subsequently creating a void to fill.

1. A Method of resolving addiction in two Phases, where first physical addiction is resolved and subsequently psycho-behavioral dependency is resolved; and with two tools, where a substitute for receiving the addictive chemical is used in response to physical cravings, and where a placebo that mimics the addictive behavior is used in response to psycho-behavioral needs and desires.
